(19) United States
(12) Patent Application Publication
(10) Pub. No.: US 2019/0359946 A1
(43) Pub. Date: Nov. 28, 2019

(54) PASSAGING AND HARVESTING FORMULATION FOR SINGLE-CELL HUMAN PLURIPOTENT STEM CELLS

(71) Applicant: LONZA WALKERSVILLE, INC., Walkersville, MD (US)

(72) Inventors: Ying NIE, Frederick, MD (US); Jonathan Allen ROWLEY, Walkersville, MD (US); Thomas FELLNER, Frederick, MD (US); Patrick WALSH, Gaithersburg, MD (US); Behnam AHMADIAN BAGHBADERANI, Walkersville, MD (US)

(21) Appl. No.: 16/478,461

(22) PCT Filed: Jul. 17, 2018
(86) PCT No.: PCT/US2018/014032

§ 371 Date: Jul. 16, 2019

Related U.S. Application Data
(60) Provisional application No. 62/447,262, filed on Jan. 17, 2017.

(51) Int. Cl. C12N 5/074

(52) U.S. Cl. CPC C12N 5/0696; C12N 2513/00; C12N 2500/60; C12N 2500/05

(57) ABSTRACT

The field of the invention is cellular and molecular biology and stem cells. Specifically, the disclosure is directed to a formulation for harvesting and passaging single cell human pluripotent stem cells comprising: (i) 1 mM to about 30 mM sodium citrate; (ii) a salt comprising 10 mM to 170 mM KCl or NaCl; and (iii) Ca2+/Mg2+-free Dulbecco's phosphate buffered saline (DPBS), wherein said formulation has an osmolarity of about 100 mOsmol/liter to about 350 mOsmol/liter. The formulation can be used for serial passaging and dislodging of pluripotent stem cells attached to 2D tissue culture vessels or grown in 3D suspension culture (small scale and large scale bioreactors) or any other application where passaging in the form of single cell population of stem cells is needed.

### FIELD OF THE INVENTION

The field of the invention is cellular and molecular biology and stem cells. Specifically, the disclosure is directed to a formulation for harvesting and passaging single cell stem cells, e.g., human pluripotent stem cells, comprising: (i) 1 mM to about 30 mM sodium citrate; (ii) a salt comprising 10 mM to 170 mM KCl or NaCl and (iii) Ca2+/Mg2+-free Dulbecco's phosphate buffered saline (DPBS), wherein said formulation has an osmolarity of about 100 mOsmol/liter to about 350 mOsmol/liter.

### BACKGROUND OF THE INVENTION

Human pluripotent stem cells (hPSCs), including human embryonic stem cells (hESCs) and induced pluripotent stem cells (iPSCs), can proliferate indefinitely in culture while maintaining the capability to differentiate into multiple types of somatic cells. These cells are greatly valued as providing unlimited cell source in cell therapy and regenerative medicine. As demonstrated by recent FDA approval of clinical trials, human embryonic stem cell (hESC)-based cell therapies are progressing from bench to clinic. However, currently available traditional tissue culture flasks and T-flask-based culture platforms severely limit the scalability of hPSCs production. To unleash the potential of hPSCs in cell therapy and regenerative medicine, a scalable hPSC manufacturing process must be developed. Scaling up existing flask-based processes is a critical stepping stone in translating current hPSC research into clinical application. One of the biggest challenges is to establish a scalable passaging method for large scale 3D suspension culture or multilayer vessels that maintains high yield, pluripotent phenotype, and karyotypic stability.

Human PSCs cells can be individualized, i.e., become single cells rather than clusters, during passaging to achieve even distribution and uniform treatments for imaging, cell sorting, and/or homogenous cell aggregate formation in suspension cultures. Cell recovery and cell number as well as viability can be critical to the success these processes. Various formulations (e.g. enzymatic dissociation method) have been developed to enable maximum cell viability when performing single cell passaging. However, hPSCs survive poorly after individualization (i.e., being made single cell), because these cells are more sensitive to treatments and are prone to cell death, a fact that has made the development of a universal dissociation method particularly challenging. Most importantly, some of the existing single cell dissociation methods (e.g. enzymatic dissociation) are known to impact the cellular characteristics or genetic stability of the cells because of cleaving off important cell adhesion and cell-cell interaction mediators from cell surface during the treatment. The quality of culture conditions is also crucial to the maintenance and expansion of the hPSCs. The medium components related to feeder cells or animal products often greatly affect the consistency of the cell culture, which could be even more problematic when cells have potential applications in translational research.

Like traditional approaches for passaging clusters, passaging of single cell hPSCs are often chosen based on cell survival and/or sensitivity. Traditionally, hPSCs are usually passaged as aggregates with enzymatic dissociation, with collagenase used for culture on feeder cells (Thomson J A, et al., Science. 282:1145-1147 (1998); Reubinoff B E, et al., Nat Biotechnol. 18:399-404 (2000)) and Dispase used for culture on feeder-free cells (Ludwig T E, et al., Nat Methods. 3:637-646 (2006)). Mechanical approaches, such as cell scrapers and other passaging tools, have also been developed to dissociate cells as aggregates. These processes are labor intensive and cannot be applied in culturing hPSCs in multilayer cell culture vessels, the platform widely used in producing commercial scale adherent cells. Cells growing in multilayer cell culturing vessels cannot be accessed for scraping. In addition, mechanical scraping may cause severe damage to cells. Without scraping, cell viability can increase up to 90 percent.

In differentiation or transfection experiments, TrypLE™ and ACCUTASE® can be used to individualize hPSCs, but poor survival often leads to abnormal karyotypes (Ellerstrom C, et al., Stem Cells. 25:1690-1696 (2007); Bajpai R, et al., Mol Reprod Dev. 75:818-827 (2008); Thomson A, et al., Cloning Stem Cells. 10:89-106 (2008)). Often, small chemicals, such as Rho-associated protein kinase (ROCK) inhibitors, must be used to boost cell survival in this process (Watanabe K, et al., Nat Biotechnol. 25:681-686 (2007)).

All these methods require specialized tools or reagents that are costly for long-term or large-scale experiments. At the same time, the consistency of enzymatic methods is usually affected by the quality of enzymes from batch to batch. Given the variability of these methods, it is highly desirable to find a safe, consistent and reproducible approach that lacks the use of enzymes and can maintain the critical characteristics of pluripotent stem cells without impacting genetic stability of the cells.

Recently, passaging hESCs with non-enzymatic cell detachment solutions, mainly EDTA (ethylene diamine tetraacetic acid) solutions, has been adopted by some hPSC labs and is spreading from academic labs into industry. One of the commercially available EDTA-containing solutions for cell dissociation is VERSENE® EDTA, which contains 0.55 mM EDTA and has been used for harvesting and passaging hPSCs. The typical procedure of passaging hESCs with VERSENE® EDTA starts with washing the culture with Ca²⁺/Mg²⁺-free buffer (for example, Dulbecco's phosphate-buffered saline; DPBS), followed by incubating the culture in VERSENE® EDTA for 4 to 9 minutes. VERSENE® EDTA is then removed and cells are physically removed from the surface as clusters by manual hosing of the cells with culture medium via pipetting. Compared with the conventional enzymatic-treatment-followed-by-scraping method (see Table 1), the advantage of this method is that (1) it uses a non-enzymatic solution—thus, there is no need for post-detachment washing or centrifugation to eliminate enzyme, and (2) it does not require scraping—the cells treated with VERSENE® EDTA can be washed off the surface. As described in Table 1, the hESCs treated with VERSENE® EDTA and detached without scraping have higher post-detachment viability and re-attach to the new culturing surface much faster (minutes vs. hours) when passaged.

TABLE 1
Methods of Harvesting/Passaging hESCs

| Conventional Enzymatic and Scraping Method | | VERSENE ® EDTA Method | |
|---|---|---|---|
| 1. | Remove culture medium | 1. | Remove culture medium |
| 2. | Incubate in collagenase or Dispase ® at 37° C. for | 2. | Was once with Ca2+/MG2+-free |
| | 2-5 minutes | | buffer (for example, DPBS) |
| 3. | Remove collagenase or Dispase ® | 3. | Incubate in VERSENE ® EDTA at |
| | | | room temperature for 4-9 minutes |
| 4. | Wash three times with culture medium | 4. | remove VERSENE ® EDTA |
| 5. | Scrape hESCs off the surface in culture medium | 5. | hose the cells off the surface with |
| | with cell scraper | | culture medium |
| 6. | Collect the colony clumps (harvest) or transfer | 6. | collect the cell clusters (harvest) or |
| | into a fresh culture vessel (passage) | | transfer into fresh culture vessel |
| | | | (passage) |

However, when applied into expanding hESC in multilayer vessels, the VERSENE® EDTA passaging/harvesting method is not ideal. VERSENE® EDTA seems to breaks down cell-cell association faster than it breaks cell-surface bonding. After the removal of VERSENE® EDTA, in six-well plate or T-flask culture format, fluidic shear force generated by hosing with culture medium via manual pipetting is needed to dislodge the cells off the surface. However, hESC culture in multilayer vessels cannot be manually sheared with culture medium as pipettes cannot be introduced inside the vessels. Instead, in this culture format, after VERSENE® EDTA is replaced with culture medium, vigorous tapping is applied to dislodge the cells. Mechanical force (tapping) follows replacement of VERSENE® EDTA with culture medium immediately because VERSENE® EDTA treated hESCs quickly re-attach to the surface once they come in contact with culture medium. In fact, with the current state-of-art, it is only possible to harvest 40-70% of the entire culture in multilayer cell factories—dramatically impacting the yield of these very expensive cells. One possible solution to increase the yield is not to replace VERSENE® EDTA with culture medium and to dislodge the cells in the presence of VERSENE® EDTA instead. However, in this case, the exposure time of cells to VERSENE® EDTA is increased, which increases the risk of obtaining karyotypic unstable colonies. In addition, extra steps of post-detachment processing follow the withdrawal and neutralization of VERSENE® EDTA from the final harvest, which adds to the labor intensity. Finally, passaging using VERSENE® EDTA treatment is not a viable option when serial passaging of PSCs in 2D or 3D as single cells are needed because PSCs stay in cell aggregates or colonies upon exposure to VERSENE® EDTA.

Various publications are cited herein, the disclosures of which are incorporated by reference herein in their entireties.

### SUMMARY OF THE INVENTION

The present disclosure is directed to harvesting and passaging formulations for human stem cells, e.g., pluripotent stem cells, and uses of such formulations. In some embodiments, the disclosure is directed to a formulation for harvesting and passaging single cell stem cells, e.g., human pluripotent stem cells, comprising: (i) 1 mM to about 30 mM sodium citrate; (ii) a salt comprising 10 mM to 170 mM KCl or NaCl; and (iii) Ca2+/Mg2+-free Dulbecco's phosphate buffered saline (DPBS), wherein said formulation has an osmolarity of about 100 mOsmol/liter to about 350 mOsmol/liter.

In some embodiments, the osmolarity of the formulation is of about 200 mOsmol/liter to about 300 mOsmol/liter. In some embodiments, the osmolarity of the formulation is of about 250 mOsmol/liter to 300 mOsmol/liter.

In some embodiments, the sodium citrate is at a concentration of about 5 mMol/liter to about 15 mMol/liter.

In some embodiments, the salt is KCl. In some embodiments, the KCl is at a concentration of about 40 mMol/liter to about 150 mMol/liter. In some embodiments, the KCl is at a concentration of about 80 mMol/liter to about 120 mMol/liter.

In some embodiments, the formulation has a pH of about 7 to about 8. In some embodiments, the formulation has a pH of about 7.4 and 7.8. In some embodiments, the formulation is substantially free of enzymes.

In some embodiments, the formulation further comprises a human stem cell, e.g., a human pluripotent stem cell. In some embodiments, the human pluripotent stem cell is selected from the group consisting of embryonic stem cell, somatic stem cell, and induced pluripotent stem cell. In some embodiments, the human stem cell is an induced pluripotent stem cell. In some embodiments, the human stem cell is a tissue-specific stem cell selected from the group consisting of an epidermal stem cell, blood stem cell, hematopoietic stem cell, epithelial stem cell, cardio stem cells, and neural stem cells.

In some embodiments, the disclosure is directed to a method for harvesting and subsequent passaging of human pluripotent stem cells (hPSCs) comprising: incubating the hPSCs in the formulations as described herein in a cell culture plate or vessel for about 2 minutes to about 20 minutes, wherein said hPSCs detach from the cell culture plate or vessel as single cells having cell viability of about 85% and about 100%. In some embodiments, the cell culture plate or vessel is selected from the group consisting of a petri dish, multi-well cell culture plate, stacked cell culture apparatus, cell culture factory, or conical tube. In some embodiments, the hPSCs are incubated in a Bioreactor, 3D suspension culture vessel, or conical tube. In some embodiments, the method further comprises downstream processing of the single cells, wherein downstream processing is selected from the group consisting of continuous counter-flow centrifugation technology, formulation, automated vialing, cryopreservation, and high-throughput screening, genetic editing, and directed differentiation.

In some embodiments, the disclosure is directed to a method of optimizing a single-cell passaging solution for human pluripotent stem cells, comprising: (i) creating a plurality of single-cell passaging solutions, each of the single-cell passaging solutions comprising at least one Ca²⁺ chelator and a known osmolarity, and wherein each of the single-cell passaging solutions in the plurality of the single-cell passaging solutions have varying concentrations and varying osmolarities, (ii) testing each of said plurality of single-cell passaging solutions to determine percentage of culture detached at a given treatment time and percentage of single cells at each given concentration of Ca²⁺ chelator and osmolarity, and (iii) selecting a preferred single-cell passaging solution from the plurality of single-cell passaging solutions.

In some embodiments, the disclosure is directed to a single-cell passaging solution obtained by the methods described herein.

In some embodiments, the disclosure is directed to a method for harvesting and subsequent passaging of single-cell hPSCs, comprising passaging the hPSCs with the formulations as described herein, at a split ratio of 1:5 to 1:60, wherein the culture reaches confluence within seven days after split.

In some embodiments, the disclosure is directed to a method for harvesting and subsequent passaging of human pluripotent stem cells (hPSCs) comprising: (i) plating the hPSCs in medium, (ii) aspirating the medium, (iii) washing the hPSCs with DPBS, (iv) adding the formulations described herein to the hPSCs and incubating for 1 minute to 30 minutes, and (v) resuspending the hPSCs in culture media. In some embodiments, the formulation of (iv) is removed prior to resuspending the hPSCs in culture media.

In some embodiments, the disclosure is directed to a method for harvesting and subsequent passaging of human pluripotent stem cells (hPSCs) grown in the form of cell aggregates in 3D suspension bioreactor comprising: (i) culturing hPSCs in the form of cell aggregates in medium using a suspension culture bioreactor, (ii) separating and removing the hPSCs from the medium, (iii) washing the hPSCs with DPBS, (iv) adding a formulation as described herein, agitating gently, and incubating for 1 minute to 50 minutes, and (v) resuspending the hPSCs in culture media. In some embodiments, the formulation of (iv) is removed prior to resuspending the hPSCs in culture media.

### BRIEF DESCRIPTION OF THE DRAWINGS

FIG. 1 is an overview of the generation of functional human pancreatic β cells in vitro (Pagliuca et al., Cell 159:428-439 (2014)) following a directed differentiation process starting from pluripotent stem cells at stage 0 and induction into definitive endoderm, pancreatic progenitor cells, endocrine progenitor, and finally insulin secreting beta islet cells.

FIG. 2 is a schematic of the experimental procedure for thawing pluripotent stem cells and expansion in 2D (i.e. well plate or tissue culture flask) using different cell culture system (including medium, matrix, and passaging solution) and then passaging hPSCs into a 3D vessel (Biott Spinner).

FIG. 3 is the results in planar culture of WA27 stem cells cultured in (i) ESSENTIAL 8® media (Thermofisher)+recombinant vitronectin (rVTN), (ii) NUTRISTEM® media (Biological Industries)+Laminin and E-Cadherin L&E-Cad, (iii) L7™ Cell Culture system including L7™ Media (Lonza)+L7™ Matrix (Lonza), (iv) mTeSR™1 media (Stemcell Technologies)+L7™ matrix (Lonza), or (v) ESSENTIAL 8® (Thermofisher)+(rVTN). The cells were then passaged using VERSENE® EDTA solution (Lonza), TrypLE™ solution (ThermoFisher), or the Formulation 3 (“L7F3”) as described in Table 2. The cells were visualized after Day 1 at 4× magnification. P24 means Passage 24 & T-75 means Tissue culture flask—T-75

FIG. 4 is the results in planar culture of WA27 stem cells cultured in (i) ESSENTIAL 8® Medium (Thermofisher)+recombinant vitronectin (rVTN), (ii) NUTRISTEM® media (Biological Industries)+Laminin and E-Cadherin (L&E-Cad), (iii) L7™ Cell Culture system including L7™ Media (Lonza)+L7™ Matrix (Lonza), (iv) mTeSR™1 media (Stemcell Technologies)+L7™ matrix (Lonza), or (v) ESSENTIAL 8® (Thermofisher)+rVTN. The cells were then passaged using VERSENE® EDTA solution (Lonza), TrypLE™ solution (ThermoFisher), or the Formulation 3 (“L7F3”) as described herein. The cells were visualized after Day 3 at 4× magnification.

FIG. 5 depicts the results of H1 cells inoculated at a concentration of about 0.6×10⁶ cells/mL in Nutristem medium in Biott Spinner culture after serial sub-culturing of the cells in 2D tissue culture flasks in (i) ESSENTIAL 8®+rVTN matrix and passaged with TrypLE™, or (ii) ESSENTIAL 8®+rVTN matrix and passaged with Formulation 3 (“L7F3”). During the cell expansion in 2D culture, the E8 medium was supplemented with basic Fibroblast grown factor (bFGF) at 100, 40 or 10 ng/mL. The cells in suspension culture were serially sub-cultured with Formulation 3 (“L7F3”). The cells were visualized after Day 4.

FIG. 6 depicts the results of directed differentiation of H1 cells following expansion in 2D (tissue culture flask) and 3D suspension culture (Biott Spinner) in different cell culture media as described in FIG. 5. Depending on cell culture condition, the cells demonstrate morphology of the cells resembling pancreatic progenitor cells at stage 4 of differentiation. This image demonstrates that the cells grown in suspension and passaged using Formulation 3 “L7F3” maintain the capacity to differentiate into specific cell lineage (in this case endoderm).

FIG. 7 depicts flow cytometry analysis of expression of various transcriptions factors (Oct-4, Sox-17, PDX-1, and NKX6.1) for H1 cells following expansion in 2D (tissue culture flask), 3D suspension culture (Biott Spinner) in different cell culture media as described in FIG. 5, and then directed differentiation into pancreatic progenitor cells. The cells grown in suspension and passaged using Formulation 3 “L7F3” maintain the capacity to differentiate into high level pancreatic progenitor cells exhibiting high level of double positive expression of PDX-1 and NKX6.1 in the absence of pluripotent stem cell marker Oct4 and early endoderm marker SOX-17. Once again, the expression of PDX-1 and NKX6.1 confirms that the cells grown in suspension and passaged using Formulation 3 “L7F3” maintain the capacity to differentiate into a specific cell lineage.

FIG. 8 depicts images of pluripotent stem cells aggregates dissociated into single cell suspensions cultures in the 3D culture (Biott Spinner) containing Formulation 3 (“L7F3”) using agitation and without manual pipetting. The cells were initially inoculated at 0.6×10⁶ cells/mL in suspension culture and grew in the form of cell aggregates. After removing the cell culture medium, the cell aggregates were exposed to L7F3 passaging solution for different incubation time (20 min, 30 min, and 40 min) while staying in suspension through agitation at 60 rpm.

### DETAILED DESCRIPTION OF THE INVENTION

Formulations and methods are disclosed for the passaging of human stem cells (hSCs), e.g., human pluripotent stem cells (hPSCs), into single cells without the use of enzymes and/or scraping to dislodge cells from cell culture vessels. The formulations and methods permit the harvesting of cells as single cells from the surface of various cell culture vessels including well plates or tissue culture flasks as well as hPSCs grown in 3D cell culture vessels. Further, the formulations and methods provide high yields of harvested cells for subsequent passaging and high post-harvest cell viability. Pluripotent stem cells passaged with the formulations and methods described herein remain undifferentiated and express typical stem cell markers, while, at the same time, retain their differentiation capability and can differentiate into the cells in all three germ layers and generate teratomas, even after numerous rounds of harvesting and passaging. These hPSCs also maintain normal karyotype after passaged with the formulations for extended periods of time.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the method/device being employed to determine the value, or the variation that exists among the study subjects. Typically, the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

The use of the term “or” in the claims is used to mean “and/or” unless explicitly indicated to refer only to alternatives or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited, elements or method steps. It is contemplated that any embodiment discussed in this specification can be implemented with respect to any method, system, host cells, expression vectors, and/or composition of the invention. Furthermore, compositions, systems, host cells, and/or vectors of the invention can be used to achieve methods and proteins of the invention.

The use of the term “for example” and its corresponding abbreviation “e.g.” (whether italicized or not) means that the specific terms recited are representative examples and embodiments of the invention that are not intended to be limited to the specific examples referenced or cited unless explicitly stated otherwise.

The present invention provides a non-enzymatic passaging formulation and a method of harvesting and subsequently passaging pluripotent stem cells from both 2D tissue culture vessel and 3D suspension culture as single cells with high yield and high post-passaging cell viability. The formulations of the present disclosure provide a scalable and high-yielding passaging and harvesting formulation and method for hPSCs that eliminates or reduces the drawbacks of methods known in the art.

The present disclosure is directed to harvesting and passaging formulations for human pluripotent stem cells and uses of such formulations. In some embodiments, the disclosure is directed to a formulation for harvesting and passaging single cell human pluripotent stem cells comprising: (i) 1 mM to about 30 mM sodium citrate; (ii) a salt comprising 10 mM to 170 mM KCl or NaCl; and (iii) Ca2+/Mg2+-free Dulbecco's phosphate buffered saline (DPBS), wherein said formulation has an osmolarity of about 100 mOsmol/liter to about 350 mOsmol/liter.

The term “stem cells” refers to cells that have the capacity to become at least all differentiated cell types of their lineage in that tissue. Stem cells can have two important characteristics that distinguish them from other types of cells. First, they are unspecialized cells that renew themselves for long periods through cell division. Secondly, under suitable conditions they can be induced to become cells with special functions, which may be considered differentiated. As used herein the term “human stem cell” refers to a human cell that can self-renew and differentiate to at least one cell type. The term “human stem cell” encompasses human stem cell lines, human pluripotent cells (including human and human-derived induced pluripotent stem cells and embryonic stem cells), human multipotent stem cells or human adult stem cells. A “pluripotent stem cell” can include a stem cell that can give rise to all three germ layers, i.e., endoderm, mesoderm, and ectoderm. As used herein, the term “adult stem cell” refers to a stem cell derived from a tissue of an organism after embryonic development is complete, i.e., a non-embryonic stem cell; such cells are also known in the art as “somatic stem cells.” In some embodiments, the human pluripotent stem cell is an embryonic stem cell or an induced pluripotent stem cell. In some embodiments, the human stem cell is an induced pluripotent stem cell. In some embodiments, the human stem cell is a tissue-specific stem cell selected from the group consisting of an epidermal stem cell, blood stem cell, hematopoietic stem cell, epithelial stem cell, cardio stem cells, and neural stem cells.

Stem cells can be derived from various tissues. For example, stem cells may be from ectoderm (epidermal, neural, neural crest, and hair follicle); mesoderm (cardiac muscle, skeletal muscle, umbilical cord blood, mesenchymal, hematopoietic, umbilical cord matrix, and multipotent adult precursor); endoderm (pancreatic islet and hepatic oval); and germ (primordial germ) stem cells. In some embodiments, the human stem cell is a human mesenchymal stem cell. In some embodiments, the stem cell is a pluripotent stem cell. In some embodiments, the stem cell is an induced pluripotent stem cell. In some embodiments, the human pluripotent stem cell is derived from a fibroblast or peripheral blood derived mononuclear cell, or cord blood derived progenitor cell, or bone marrow derived stem or progenitor cell.

In some embodiments, the disclosure is directed to a formulation for passaging single human induced pluripotent stem cell (iPSC), which is a stem cell type generated by contacting a human somatic cell with induction factor that reprograms the somatic cell to generate an iPSC. The induction factor includes at least one “reprogramming element”, that is, an element that directs the somatic cell to de-differentiate, and an “expression-enabling element”, which enables entry and/or expression of the reprogramming element within the somatic cell. The induction factor can be a genetic construct or a fusion protein.

Where the induction factor is a genetic construct, the construct can bear one or more nucleotide sequences encoding one or more reprogramming elements selected from OCT4, SOX2, NANOG, LIN28, and C-MYC and a Notch pathway molecule, or an active fragment or derivative thereof. The construct may encode multiple reprogramming elements, or only a single reprogramming element. The single reprogramming element can encode one of OCT4, SOX2, LIN28, C-MYC or NANOG. Alternatively, the construct can include two reprogramming elements, selected from OCT4 and SOX2, or OCT4 and NANOG, or SOX2 and NANOG, OCT4 and LIN28, or LIN28 and NANOG, or SOX2 and LIN28. The construct may further comprise any combination of two or more reprogramming elements, selected from OCT4, SOX2, NANOG, LIN28, and C-MYC and a Notch pathway molecule. The expression-enabling element of the genetic construct can be a lentiviral or episomal vector backbone.

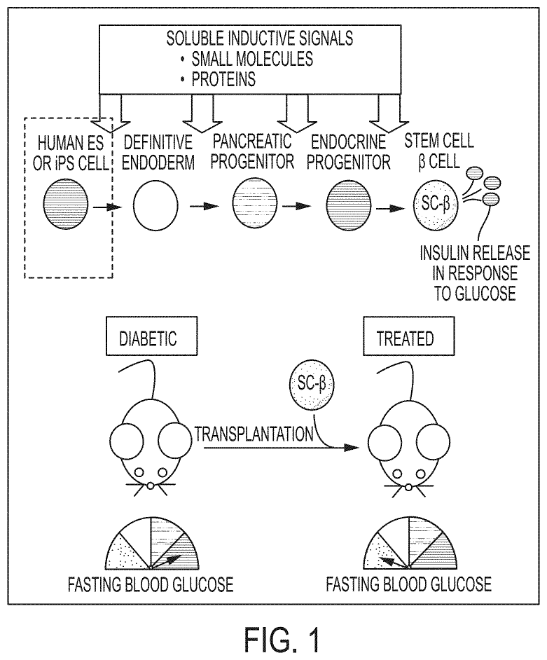

The culture of human pluripotent stem cells shares many of the same protocols as standard mammalian cell culture. However, successful culture and maintenance of human pluripotent stem cells (hPSCs) in an undifferentiated state requires additional considerations to ensure that cells maintain their key characteristics of self-renewal and pluripotency. There are several basic techniques needed for the culturing of mammalian cells, including thawing frozen stocks, plating cells in culture vessels, changing media, harvesting, passaging and cryopreservation. As example for application human pluripotent stem cells in generation of functional/specialized cells, an overview of the growth and differentiation of human stem cells to be use for a therapeutic use can be found in FIG. 1. Harvesting refers to the collecting of the stem cells for their intended use, e.g., a therapeutic use. Passaging refers to the removal of cells from their current culture vessel and transferring them to one or more new culture vessels. Passaging is necessary to reduce the harmful effects of overcrowding and for expansion of the culture. In some embodiments, passaging includes the removal of the cells from their current vessel by dislodging cells adhered to the vessel before transferring the cells to the new vessel. In some embodiments, passaging includes the removal of pluripotent stem cell aggregates from their current suspension culture (e.g., a 3D culture or bioreactor) by removing the cell culture medium, exposing the cell aggregates to the passaging solution, agitation or mixing, and dissociating the aggregates into single cells.

Different cell lines have different growth kinetics and thus the time and conditions for passaging varies among different cell lines. However, generally hPSCs grow slowly during the first couple of weeks after being thawed, then faster until the growth rate reaches a plateau. The cell growth rate then can stay in that plateau for many passages if cells are cultured properly. In some embodiments, the growth of the stem cells of the present invention are observed daily to establish the growth pattern of the cell line being cultured.

In some embodiments, cell growth and quality are evaluated under a microscope. In some embodiments, visual observations (via microscope) can be used to determine when and how often the cells are passaged. In 2D tissue culture vessels, e.g., 2D tissue culture flasks, the cells attach to the surface of the culture vessel previously coated with one or more proteins including Vitronectin, Laminin, cadherin, or other cell substrates known in the field. In 2D culture, healthy, undifferentiated hPSC colonies generally have well-defined uniform borders and the individual cells within the colony appear to be similar. The exact colony morphology will differ with different cell lines and culture conditions (e.g., the culture used). As used herein, the term “morphology” is used to describe one or more characteristics regarding the physical appearance of a cell that distinguishes it from or renders it similar to a given cell type or state. In some embodiments, the cells adhere to one another and form aggregates of spherical or rounded shape in suspension culture (3D bioreactor) in the absence of cell-surface attachment. The term “morphology” in 3D culture refers to the aggregates of cells.

Human pluripotent stem cells generally survive poorly after individualization (i.e., being made single cell), because these cells are sensitive to treatments and are prone to cell death, a fact that has made the development of a universal dissociation method particularly challenging. Various formulations have been attempted to maximize cell viability when performing single cell passaging and allow the expansion of the pluripotent stem cells. However, these formulations often have animal products that can affect the consistency of the cell culture, which could be even more problematic when cells have potential applications in translational research. Some methods used for dissociation in the passaging step for pluripotent stems cells include enzymatic dissociation with a collagenase or dispase (Stem Cell Technologies), or the use of TrypLE™ and ACCUTASE® (which often leads to genetic instability or abnormal karyotypes), mechanical approaches, such as cell scrapers and trituration using pipette, which often leads to significant cell death and poor viability and yield after passaging). In some embodiments, the disclosure is directed to a formulation for harvesting and passaging single human pluripotent stem cells, wherein the formulation is substantially free of an animal product. In some embodiments, the disclosure is directed to a formulation for harvesting and passaging single human pluripotent stem cells, wherein the formulation is substantially free of an enzyme. In some embodiments, the formulation is substantially free of collagenase, dispase, TrypLE™ and/or ACCUTASE®. In some embodiments, the disclosure is directed to a formulation for harvesting and passaging single human pluripotent stem cells, wherein the formulation is substantially free of Rho-associated protein kinase (ROCK) inhibitors. In some embodiments, the disclosure is directed to a formulation for harvesting and passaging single human pluripotent stem cells, wherein the formulation is substantially free of EDTA.

Different culture conditions yield different types of differentiated cells and varying rates of growth. In some embodiments, the stem cells are passaged when any of the following occur: (i) the thawed cells are 7 days, 10 days, 14 days, 15 days, 20 days, or 21 days old, (ii) when greater than about 30%, greater than about 40%, greater than about 50%, greater than about 60% or greater than about 70% of the colonies are greater than 2000 μm, (iii) colonies are too dense (at approximately greater than about 50%, greater than about 60%, greater than about 70%, or greater than about 80% confluence), (iv) the cells form aggregates of the cells larger than 50 μm, larger than 100 μm, larger than 150 μm, larger than 200 μm, larger than 250 μm, larger than 300 μm, larger than 350 μm, larger than 400 μm, larger than 450 μm, larger than 500 μm in suspension culture, or (v) colonies exhibit increased differentiation.

In some embodiments of the present invention, the stem cells described herein survive passaging. As used herein, the term “survives passaging” refers to the ability of a single cell to survive passaging from a parent culture to a sub-culture using the formulations described herein. In some embodiments, greater than 60%, greater than 70%, greater than 80%, greater than 85%, greater than 90%, greater than 95%, greater than 96%, greater than 97%, greater than 98% or greater than 99% of the cells survive passaging, i.e., remain viable.

As described herein, formulations have been found which aid in the harvesting and passaging of single cell human pluripotent stem cells. The formulation described herein comprise sodium citrate. The term sodium citrate can include any of the sodium salts of citrate, including the monosodium salt, disodium salt, and trisodium salt, as well as the sodium and the weak acid citrate, when found in solution. One of skill in the art can appreciate that other Group I salts, e.g., lithium and potassium, can also be used and would be considered equivalents to a sodium salt.

While not being bound by any theory, sodium citrate may disrupt the cell-surface bond and cell-cell association by chelating/sequestering Ca²⁺, the divalent cation required for cell-surface and cell-cell binding. The sodium citrate-based formulations and methods as described herein were designed and developed to address the unique challenges in routine or scale up hPSC culture and manufacturing processes. hPSCs are normally passaged as multi-cellular clusters/aggregates. However, in some embodiments, passaging hPSCs as single-cells is desired including (i) serial subculturing of the cells in suspension culture when single cell suspension is critical to production of large number of round cell aggregates with homogenous size distribution (the size of aggregates remain in a close size range), (ii) when single cell population of cells is needed for ease of enumeration or processing through cell characterization instruments such as flow cytometry machine or cell sorting machine, and/or (iii) start of downstream processing such as directed differentiation process with single cell population of pluripotent stem cells. On the other hand, single cell passaging is often avoided due to low cloning efficiency of hPSCs and the high risk of karyotypic abnormality. The formulations and methods described herein are optimized for harvesting and passaging single cell hPSCs in reference to some key quality parameters, for example, viability, yield, post-detachment cluster size, passageability, and ability to maintain a pluripotent phenotype. The formulations and methods described herein can be used in routine lab practice to expand hPSC cultures with reduced labor intensity and process time. For example, the formulations and methods described herein require reduced mechanical scraping (or no scraping) to get the cells off the vessel surface and the cell harvest does not need to be washed and centrifuged to remove the agents used to detach the culture. In some embodiments, the formulations and methods described herein are suitable for large-scale hPSC production when the cells are growing in multilayer cell culture vessels where scraping cannot be applied. In some embodiments, more than 90% of hPSCs grown in multilayer cell culture vessels can be harvested with more than 90% viability. In some embodiments, the formulations and methods described herein are suitable for serial subculturing of hPSCs when grown in the form of cell aggregates in scalable 3D suspension culture and dissociation into single cell suspension. In some embodiments, more than 90% of hPSCs grown in 3D culture can be harvested with more than 90% viability.

In some embodiments, additional passaging and harvesting formulations are provided including formulations containing EDTA and EGTA, other Ca²⁺ chelators besides sodium citrate, or combinations of various Ca²⁺ chelators. All of these reagents (EDTA, EGTA and sodium citrate) are Ca²⁺ chelators and have been used historically for detaching adherent cells in culture. As mentioned previously, VERSENE® EDTA has been used routinely for harvesting/passaging hESCs in some labs; both EDTA and EGTA (in combination with trypsin) were used to passage hESCs in a study published by Thomson et al. at Roslin Institute in Scotland in 2008 (Thomson et al. (2008), “Human Embryonic Stem Cells Passaged Using Enzymatic Methods Retain a Normal Karyotype and Express CD30”, Cloning and Stem Cells, 10 (1), 1-17). However, in some embodiments, passaging formulations comprising EDTA (or EGTA) increases the risk of obtaining karyotypic unstable colonies. In some embodiments, extra steps of post-detachment processing follow the withdrawal and neutralization of a passaging formulation comprising EDTA (or EGTA) from the final harvest, which adds to the labor intensity. In some embodiments, the disclosure provides for a harvesting and passaging formulation that does not contain EDTA and/or EGTA. In some embodiments, the disclosure provides for a harvesting and passaging formulation that comprises greatly reduced amounts of EDTA and/or EGTA, e.g., the formulation has less than 0.05 mM EDTA, less than 0.01 mM EDTA, less than 0.005 mM EDTA, or less than 0.001 mM EDTA.

Formulations as found herein suitable for providing single cell hPSCs useful for passaging comprise sodium citrate at a concentration of 1 mM to about 30 mM, 2 mM to about 25 mM, 3 mM to about 20 mM, or 5 mM to about 15 mM. In some embodiments, the formulations as described herein have a concentration of about 5 mM, about 10 mM, or about 15 mM. In some embodiments, the formulation comprises sodium citrate at a concentration of about 5 mMol/liter to about 15 mMol/liter.

The formulations as described herein comprise a salt. In some embodiments, the salt is a potassium chloride (KCl), sodium chloride (NaCl) salt or a combination thereof. In some embodiments, the salt comprises NaCl, KCl, LiCl, Na₂HPO₃, NaH₂PO₃, K₂HPO₃, KH₂PO₃, and/or NaHCO₃.

Formulations as found herein suitable for providing single cell hPSCs useful for passaging comprise NaCl or KCl at a concentration of 10 mM to 170 mM, 20 mM to 150 mM, 30 mM, to 130 mM, or 40 mM to 120 mM. In some embodiments, the salt is KCl. In some embodiments, the KCl is at a concentration of about 40 mMol/liter to about 150 mMol/liter. In some embodiments, the KCl is at a concentration of about 80 mMol/liter to about 120 mMol/liter. In some embodiments, the salt is NaCl. In some embodiments, the NaCl is at a concentration of about 40 mMol/liter to about 150 mMol/liter. In some embodiments, the NaCl is at a concentration of about 80 mMol/liter to about 120 mMol/liter. One of skill in the art can appreciate the concentration of the salt can be adjusted to achieve the desired osmolarity. For example, if the concentration of the sodium citrate (or another component) is reduced, the amount of salt can be increased to achieve the desired osmolarity. Likewise, if the concentration of the sodium citrate (or other component) is increased, the amount of salt can be decreased to achieve the desired osmolarity.

Various osmolarities can be used in formulations of the present invention. As described herein, adjusting the sodium citrate and salt concentrations and altering the osmolarity of the formulation to about 100 mOsmol/liter to about 350 mOsmol/liter provides a passaging solution can be used to passage single cell pluripotent stem cells, rather than clusters of stem cells. In some embodiments, reducing the osmolarity as described herein results in the hPSCs dissociating the vessel more easily, e.g., without mechanical scraping. In some embodiments, the formulation has an osmolarity of about 100 mOsmol/liter to about 350 mOsmol/liter, about 125 mOsmol/liter to about 320 mOsmol/liter, about 150 mOsmol/liter to about 300 mOsmol/liter, about 175 mOsmol/liter to about 275 mOsmol/liter, or about 200 mOsmol/liter to about 250 mOsmol/liter. In some embodiments, the formulation has an osmolarity of about 250 mOsmol/liter, about 260 mOsmol/liter, about 270 mOsmol/liter, about 280 mOsmol/liter, about 290 mOsmol/liter, or about 300 mOsmol/liter. In some embodiments, the osmolarity of the formulation is of about 200 mOsmol/liter to about 300 mOsmol/liter. In some embodiments, the osmolarity of the formulation is of about 250 mOsmol/liter to 300 mOsmol/liter.

In some embodiments, formulations of the present invention comprise Ca2+/Mg2+-free Dulbecco's phosphate buffered saline (DPBS). Dulbecco's phosphate-buffered saline (DPBS) is a balanced salt solution that does not contain calcium or magnesium salts, used for a variety of cell culture applications, such as washing cells before dissociation, transporting cells or tissue samples, diluting cells for counting, and preparing reagents. Formulations without calcium and magnesium are required for rinsing chelators from the culture before cell dissociation. DPBS comprises potassium chloride (0.2 g/l), potassium phosphate monobasic anhydrous (0.2 g/l), sodium chloride (8.0 g/l) and sodium phosphate dibasic-7-hydrate (2.160 g/l).

The formulations of the present invention can have various pH levels. In some embodiments, the formulation has a pH of about 7 to about 8. In some embodiments, the formulation has a pH of about 7.4 and 7.8.

In some embodiments, the disclosure is directed to a formulation for harvesting and passaging single human pluripotent stem cells, wherein the formulation is substantially free of an animal product. In some embodiments, the disclosure is directed to a formulation for harvesting and passaging single human pluripotent stem cells, wherein the formulation is substantially free of an enzyme. In some embodiments, the formulation is substantially free of collagenase, dispase, TryPLE™ and/or ACCUTASE®. In some embodiments, the disclosure is directed to a formulation for harvesting and passaging single human pluripotent stem cells, wherein the formulation is substantially free of Rho-associated protein kinase (ROCK) inhibitors. In some embodiments, the formulation is substantially free of enzymes.

The formulations as described herein are suitable for the harvesting and passaging of single cell human pluripotent stem cells. Thus, in some embodiments, the formulation further comprises a human pluripotent stem cell. In some embodiments, the formulation further comprises a human mesenchymal stem cell. In some embodiments, the human pluripotent stem cell is selected from the group consisting of embryonic stem cell and induced pluripotent stem cell. In some embodiments, the disclosure as presented herein provides for a composition comprising the formulation (sodium citrate at a concentration of about 1 mM to about 30 mM, KCl at a concentration of about 10 mMol/liter to about 170 mMol/liter and Ca2+/Mg2+-free Dulbecco's phosphate buffered saline (DPBS)), and a human pluripotent stem cell.

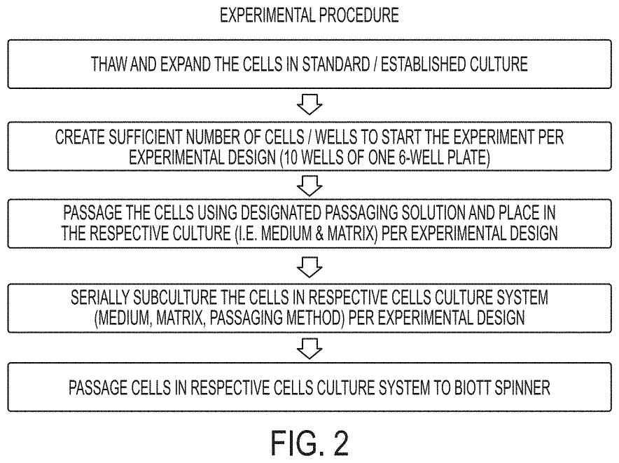

Various techniques and protocols are used for the culturing of mammalian cells, including thawing frozen stocks, plating cells in culture vessels, changing media, passaging and cryopreservation. A general overview of the culturing and harvesting process is found in FIG. 2. In some embodiments, the disclosure is directed to a method for harvesting and subsequent passaging of human pluripotent stem cells (hPSCs) in 2D comprising: incubating the hPSCs in the harvesting and passaging formulations as described herein in a cell culture plate or vessel for about 2 minutes to about 20 minutes, wherein the hPSCs detach from the cell culture plate or vessel as single cells having cell viability of about 85% and about 100%. In some embodiments, the hPSCs are incubated for about 5 minutes to about 15 minutes, or for about 8 to about 12 minutes in the harvesting and passaging formulation. In some embodiments, the disclosure is directed to a method for harvesting and subsequent passaging of human pluripotent stem cells (hPSCs) from their current suspension culture (i.e. 3D culture or bioreactor) by removing the cell culture medium, exposing the cell aggregates to the passaging solution for about 10 minutes to about 40 minutes agitation or mixing, and dissociating the aggregates into single cells,

In some embodiments, about 0.2 mL to about 10 mL of harvesting and passaging formulation is added to the cell culture plate or vessel. In some embodiments, about 0.5 mL to about 5 mL of harvesting and passaging formulation is added to the cell culture plate or vessel. In some embodiments, about 1 mL to about 2 mL of harvesting and passaging formulation is added to the cell culture plate or vessel. In some embodiments, about 5 mL to about 10 mL of harvesting and passaging formulation is added to the suspension culture vessel or Bioreactor. In some embodiments, about 15 mL to about 40 mL of harvesting and passaging formulation is added to the suspension culture vessel or Bioreactor. In some embodiments, about 50 mL to about 100 mL of harvesting and passaging formulation is added to the suspension culture vessel or Bioreactor. In some embodiments, about 150 mL to about 500 mL of harvesting and passaging formulation is added to the suspension culture vessel or Bioreactor. In some embodiments, about 500 mL to about 2000 mL of harvesting and passaging formulation is added to the suspension culture vessel or Bioreactor. The amount of harvesting and passaging formulation can be adjusted according the type and size of the vessel.

In some embodiments, the cell culture plate or vessel is tapped or swirled to assist in dislodging the cells off the surface. In some embodiments, the cell aggregates formed in 3D suspension culture are settled and growth medium is aspirated using an aspirator or medium harvest line. In some embodiments, no mechanical pipetting or scraping is utilized to dislodge the cells off the surface. In some embodiments, agitation is used in suspension culture to dissociate the cell aggregates into single cells in the presence of the passaging formulation. In some embodiments, the agitation speed in the bioreactor is 40 rpm, 50 rpm, 60 rpm, 70 rpm, 80 rpm, or 90 rpm. In some embodiments, the incubation time of the cell aggregates with passaging solution in suspension bioreactor is 10, 20, 30, 40, or 50 min. In some embodiments, growth medium is added to the harvesting and passaging solution after the incubation period. In some embodiments, the harvesting and passaging formulation as described herein is not removed before the growth medium is added. In some embodiments, the hPSCs in the harvesting and passaging formulation are centrifuged after the incubation period, and the supernatant comprising the harvesting and passaging formulation is aspirated, with the pellet resuspended with an appropriate volume of growth medium supplemented with Y-27623 (Y compound) (Rho-associated protein kinase (ROCK) inhibitor, Stemcell Technologies, Cambridge, Mass.) In some embodiments, the hPSCs in the harvesting and passaging solution are centrifuged at 100 g to 300 g, e.g., 200 g, for about 1 to about 10 minutes, e.g., about 2 minutes to 5 minutes or about 3 minutes.

Various vessels and containers (e.g., cell culture plate or vessel) are known to those in the art to be useful for culturing and passaging hPSCs. In some embodiments, the cell culture plate or vessel is selected from the group consisting of a petri dish, multi-well cell culture plate, stacked cell culture apparatus, cell culture factory, conical tube, different types of spinner flasks equipped with agitator or impeller, or suspension culture bioreactors equipped with impeller. In some embodiments, the hPSCs are incubated in a conical tube.

In some embodiments, the method further comprises downstream processing of the single cells, wherein downstream processing is selected from the group consisting of continuous counter-flow centrifugation technology, imaging, cell sorting, formulation, automated vialing, cryopreservation, high-throughput screening, genetic editing, directed differentiation, and for work in suspension cultures where cell recovery and cell number are critical to success, e.g., to serve as a basis of comparison for clone selection.

The present disclosure provides for the use of a Ca²⁺ chelator formulation, e.g., sodium citrate, with specified osmolarity suitable for the harvesting and passaging of cells. The disclosure of the present invention is suitable for optimizing this invention to find a single-cell passaging solution for human pluripotent stem cells for a specific cell type, or a specific culturing condition. In some embodiments, the disclosure is directed to a method of optimizing a single-cell passaging solution for human pluripotent stem cells, comprising: (i) creating a plurality of single-cell passaging solutions, each of the single-cell passaging solutions comprising at least one Ca′ chelator and a known osmolarity, and wherein each of the single-cell passaging solutions in the plurality of the single-cell passaging solutions have varying concentrations and varying osmolarities, (ii) testing each of said plurality of single-cell passaging solutions to determine percentage of culture detached at a given treatment time and percentage of single cells at each given concentration of Ca²⁺ chelator and osmolarity, and (iii) selecting a preferred single-cell passaging solution from the plurality of single-cell passaging solutions. In some embodiments, the disclosure is directed to a single-cell passaging solution obtained by the methods described herein.

In some embodiments, the disclosure provides a formulation and method optimized for harvesting and passaging single hPSCs based on parameters such as high viability, high yield, large post-detachment cluster size, serial passageability, and maintenance of the pluripotent phenotype (for example, expression of markers typically associated with stem cells such as OCT4, Sox2, Nanog, SSEA4, TRA-1-60 and TRA-1-81) and karyotypic stability.

In some embodiments, the disclosure is directed to a method for harvesting and subsequent passaging of single-cell hPSCs, comprising passaging the hPSCs with the formulations as described herein, at a split ratio of 1:5 to 1:60, wherein the culture reaches confluence within 3 to 10 days after split. In some embodiments, the disclosure is directed to a method for harvesting and subsequent passaging of single-cell hPSCs, comprising passaging the hPSCs with the formulations as described herein, at an inoculation cell density of 2×10⁵ cells/mL to 2×10⁶ cells/mL, wherein the culture reaches the desired cell density within 3 to 6 days after split.

In some embodiments, the disclosure is directed to a method for harvesting and subsequent passaging of human pluripotent stem cells (hPSCs) comprising: (i) plating the hPSCs in medium, (ii) aspirating the medium, (iii) washing the hPSCs with DPBS, (iv) adding the formulations described herein to the hPSCs and incubating for 1 minute to 30 minutes, and (v) adding, e.g., resuspending the hPSCs in, culture media. In some embodiments, the formulation of (iv) is removed (e.g., via filtration or centrifugation) prior to resuspending the hPSCs in culture media.

In some embodiments, the disclosure is directed to a method for harvesting and subsequent passaging of human pluripotent stem cells (hPSCs) comprising: (i) culturing hPSCs in medium using a suspension culture bioreactor, (ii) separating and removing the hPSCs from the medium, (iii) washing the hPSCs with DPBS, (iv) adding a formulation as described herein, agitating gently (e.g., at a range of 30-70 rpm), and incubating for 1 minute to 50 minutes, and (v) adding, e.g., resuspending the hPSCs in, culture media. In some embodiments, the formulation of (iv) is removed (e.g., via filtration or centrifugation) prior to resuspending the hPSCs in culture media. In some embodiments, the formulation of (iv) is not removed prior to adding the hPSCs in culture media.

In some embodiments, the disclosure provides a formulation and a method of use that can be used in routine lab practice to expand hPSC cultures with reduced labor intensity and process time.

In some embodiments, the disclosure provides a formulation and a method of use that does not require mechanical scraping to remove cells from the surface of the culture vessel and to provide single hPSCs for passaging. In some embodiments, the disclosure provides a formulation and a method of use that reduces by 50%, 80%, 90% or 95% the mechanical scraping required to remove cells from the surface of the culture vessel and to provide single hPSCs for passaging.

In some embodiments, the disclosure provides a formulation and a method of wherein the harvested cells do not need to be washed and centrifuged to remove the passaging formulation used to detach the cells from the surface of the culture vessel.

In some embodiments, the disclosure provides a formulation and a method of use wherein over 90% of hPSCs grown in planar or multilayer cell culture vessels can be harvested with over 90% viability. In some embodiments, the disclosure provides a formulation and a method of use wherein over 92%, over 94%, over 96%, or over 98% of hPSCs grown in planar or multilayer cell culture vessels can be harvested with over 90% viability. In some embodiments, the disclosure provides a formulation and a method of use wherein over 90% of hPSCs grown in planar or multilayer cell culture vessels can be harvested with over 90%, over 92%, over 94%, over 96%, over 98%, or over 99% viability. In some aspects of the embodiment, the method results in the harvest of, for example, at least 90% of the cells from the surface of the culture vessel and cell viability of at least 90%.

In some embodiments, the disclosure provides a formulation and a method of use in the process of expanding and passaging hPSCs from T-flasks into multilayer cell factories with harvesting and passaging that does not utilize any enzymes, followed by downstream processing with continuous counter-flow centrifugation technology (for example, kSep® technology).

In some embodiments, the disclosure provides a formulation and a method of use for developing a cell-detaching and cell separation formulation for hPSCs wherein the passaged cells are single cells, and the percentage of the culture detached and singularized at given treatment time can be controlled with the osmolality and Ca′ chelator concentration. Two factors identified as relating to cell detachment and cell individualization include a Ca′ chelator concentration and osmolarity.

In some embodiments, the disclosure provides a formulation and a method of use for harvesting and subsequent passaging of hPSCs grown in suspension culture (3D Bioreactor), in either a formulation disclosed herein or a formulation identified by a method disclosed herein, in cell culture vessels for two to fifty minutes allowing any hPSC aggregates to singularize or to allow the hPSCs to detach from a surface or a microcarrier, with cell viability between about 80% to 100% percent.

In some embodiments, the disclosure provides a formulation and a method of use for harvesting and subsequent passaging of hPSCs, where the hPSCs are passaged with a high split ratio (1:5 to up to 1:60; or density of cells at seeding of about 100×10³/cm² to as low as 5×10³/cm²) and the culture reaches confluence within ten days after split. In some embodiments, the disclosure provides a formulation and a method of use for harvesting and subsequent passaging of hPSCs in suspension culture, where the hPSCs are passaged at a seeding density of 2×10⁵ cells/mL to 2×10⁶ cells/mL and the culture reaches the maximum cell number within six days.

In some embodiments, the disclosure provides a formulation and a method of use for harvesting and subsequent passaging of hPSCs where the hPSCs maintain pluripotency and normal G-banding karyotype at over 50 passages.

In some embodiments, the disclosure provides a formulation and a method for selectively detaching and passaging single undifferentiated hPSCs.

In some embodiments, the disclosure provides a formulation and a method for harvesting and subsequent cryopreserving single hPSCs with high post thaw recovery and re-plating efficiency.

In some embodiments, the disclosure provides a formulation and a method of use for downstream processing of harvested single cell hPSC in a closed system including continuous counter flow, centrifugation, formulation, automated vialing and cryopreservation with controlled rate freezer.

In some embodiments, the disclosure provides a formulation and a method of use for harvesting and subsequent passaging of human pluripotent stem cells without scraping and without substantial loss of viability. In one aspect of the embodiment, the formulation includes, for example, sodium citrate, a salt, and a phosphate-buffered saline solution, at an osmolality of about 10 to 170 mOsmol/Liter.

There has thus been outlined, rather broadly, the more important features of the invention in order that the detailed description thereof that follows may be better understood, and in order that the present contribution to the art may be better appreciated. There are, of course, additional features of the invention that will be described further hereinafter.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that equivalent constructions insofar as they do not depart from the spirit and scope of the present invention, are included in the present invention.

For a better understanding of the invention, its operating advantages and the specific objects attained by its uses, reference should be had to the accompanying drawings and descriptive matter which illustrate preferred embodiments of the invention.

### EXAMPLES

### Example 1

### Preliminary Screening and Characterization of Various Non-Enzymatic Cell Detachment Formulation Solutions and Methods

A series of new passaging solutions were designed to find a solution that would assist in detaching the hPSCs from the cell wall in a single-cell state. Previously, a solution of 1 mM sodium citrate (570 mOsmol/kg) had been used. However, prior formulations resulted in the formation of clusters when the cells detached, and/or require further processing to remove the passaging formulations. The series of new passaging formulations is outlined in Table 2.

TABLE 2
New non -enzymatic single cell passaging solutions

| | | |
|---|---|---|
| Previous Formulation | 1 | mM Sodium Citrate |
| | 570 | mOsmol/kg |
| Formulation 1 | 5 | mM Sodium Citrate |
| | 270 | mOsmol/kg |
| Formulation 2 | 10 | mM Sodium Citrate |
| | 270 | mOsmol/kg |
| Formulation 3 | 15 | mM Sodium Citrate |
| | 270 | mOsmol/kg |

Each of the formulations were tested in their ability to detach hPSCs in a single-cell state while maintaining high viability. (data not shown). Formulation 3 was found to be superior to Formulations 1 and 2 based on the ability to generate larger population of single cells, fewer percentage of aggregates generated after dissociation/passaging (L7 Formulation 3 consistently generating less 5% of cell aggregates when compared to L7 Formulation 1 and 2), higher viability (L7 Formulation 3 consistently resulting in a high viability of 90% or higher when compared to L7 Formulation 1 and 2), maintaining morphology of pluripotent stem cells in culture, and robustness of the results evaluated with two different PSC lines (H1 and HEUS8). The viability and number of cell aggregates following dissociation was evaluated by running a sample taken from the cell suspension post-dissociation through Nucleocounter NC-200, which is a cell counter machine designed to evaluate the total number of cells, total number of viable cells, viability, and percentage of cell aggregates/clusters present in the sample.

### Example 2

### Comparison of Formulation 3 Against Other Dissociation Treatments in Planar Culture

The Formulation 3 passaging solution was compared with enzymatic and alternative non-enzymatic cell detachment solutions in different pluripotent stem cell lines and cell culture systems comprising of various mediums and matrices. One objective being to improve the yield of single cell hPSCs harvested from planar vessels while retaining the simplicity of previous harvesting/passaging method. This screening included three different cell lines, (H1, WA27, and HAD106), four different growth mediums (NUTRISTEM®, Biological Industries; ESSENTIAL 8® Medium (“E8 Medium”), Thermo Fisher Scientific; mTeSR™1 Medium, Stemcell Technologies; L7™ Medium, Lonza), and four different matrices (Laminin & E-cadherin; recombinant VTN; Matrigel® matrix, Corning; and L7™ Matrix, Lonza). The various combinations are outlined in Table 3 below:

TABLE 3

| | | Dissociation | |
|---|---|---|---|
| Cell line | Medium | treatment | Matrix |
| H1 | NUTRISTEM ® | TrypLE ™ | Laminin & E-Cadherin |
| | | | (per Semma protocol) |
| | E8 ® medium | Formulation 3 | rVTN |
| | mTeSR1 ™ | Formulation 3 | L7 ™ matrix |
| | L7 ™ medium | Formulation 3 | L7 ™ matrix (gradual |
| | | | change - 2 passages) |
| | mTeSR1 (control) | TrypLE ™ | Matrigel ™ |
| WA27 | NUTRISTEM ® | TrypLE ™ | Laminin & E-Cadherin |
| | | | (per Semma protocol) |
| | E8 ® medium | Formulation 3 | rVTN |
| | mTeSR1 ™ | Formulation 3 | L7 ™ matrix |
| | E8 ® medium | VERSENE ® | rVTN |
| | (control) | | |
| | L7 ™ medium | Formulation 3 | L7 ™ matrix |
| HAD106 | mTeSR1 ™ | Formulation 3 | Matrigel ® |
| | NUTRISTEM ® | Formulation 3 | L7 ™ matrix (gradual |
| | | | change - 2 passages) |
| | L7 ™ medium | Formulation 3 | L7TM matrix (gradual |
| | | | change - 2 passages) |
| | NUTRISTEM ® | TrypLE ™ | HDF |
| | (control) | | |
| | NUTRISTEM ® | TrypLE ™ | Laminin & E-Cadherin |
| | | | (per Semma protocol) |

WA27 cells were cultured on plates in the indicated medium. The cells were then removed from the culture medium by centrifugation and aspiration of the spent medium from the culture vessel. The cells were then washed once with Ca²⁺/Mg²⁺ free buffer (for example, DPBS), at 1 mL DPBS per 10 cm². 1 mL/10 cm² of pre-warmed passaging solution was added and incubated at 37° C. for 5-15 minutes. The cells were checked at 5 minute intervals. The vessel was then tapped/swirled to dislodge cells off the surface. The cell solution was then pipetted up and down five times using a 10 mL pipette. Dissociation was quenched with an equal volume of growth medium supplemented with Y compound. The cells were then centrifuged at 200 g for 3 minutes at room temperature. The supernatant was aspirated, and the cells resuspended with an appropriate volume of the designated growth medium supplemented with Y compound.

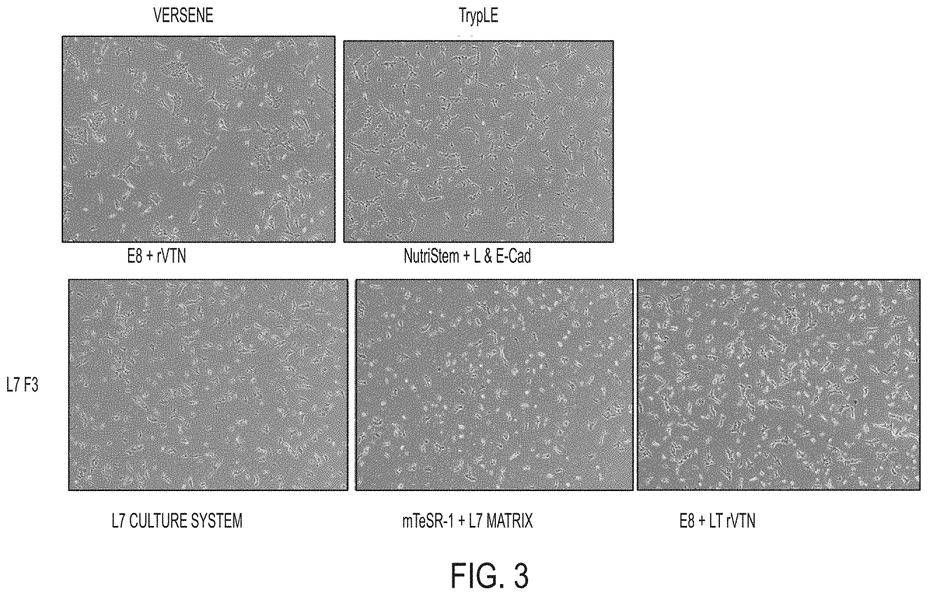

FIG. 3 shows the results of WA27 cells grown in the indicated media and matrices, and passaged using the indicated passaging formulations, including Formulation 3 of Example 1. As can be seen from the images taken on day 1 post-passaging, WA27 cells passaged using Formulation 3 passaging formulation produced comparable individualized cells and comparable or higher cell attachment when compared to enzymatic passaging TrypLE regardless of the cell culture medium (L7 medium, ESSENTIAL 8® (E8), NUTRISTEM® and mTeSR™-1 or matrix (Laminin & E-cadherin; recombinant VTN; Matrigel® matrix, or L7™ Matrix. The versene passaging solution was not able to generate a single cell suspension after passaging as it is designed for passaging of PSCs in the form of cell clusters (as shown in FIG. 3). Through this experiment, sodium citrate solution for Formulation 3 is surprisingly identified as a superior reagent compared to TrypLE™ and VERSENE® formulations.

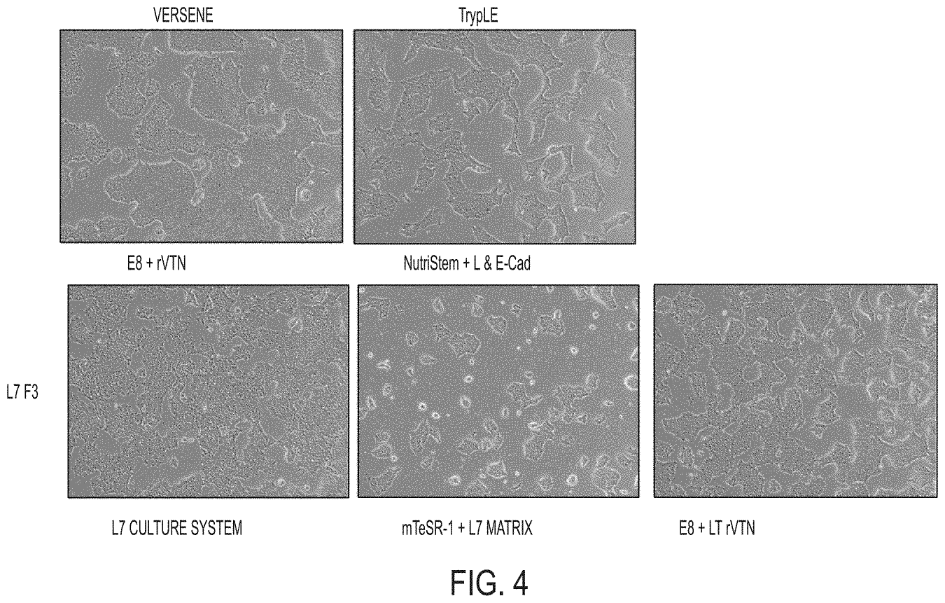

FIG. 4 show the cell growth 3 days after passaging. The use of Formulation 3 as a passaging formulation results in a significantly higher cell growth after passaging (evaluated by higher confluency in L7 cell culture system and E8 plus L7™ Matrix, relative to the use of TrypLE™ and VERSENE® passaging formulations.

A quantitative comparison between different passaging methods in different cell culture system has been demonstrated in Table 4. Formulation 3 was found to result in superior or comparable viability, total cell number, or percentage of aggregates generated after dissociation/passaging when compared to enzymatic passaging TrypLE. As expected, the Versene passaging failed to generate single cell suspension. The viability, number of cell aggregates following dissociation, and total viable cells were produced using Nucleocounter NC-200 counting, one cassette method. Considering concerns around enzymatic passaging leading to abnormal karyotype, the Formulation 3 seems to be a safer non-enzymatic passaging solution that can result in acceptable quantitative results.

TABLE 4

| | | Passaging | Converted | Converted | Total | % |
|---|---|---|---|---|---|---|
| Medium | Matrix | Formulation | VCC | Viability | Cells | Aggregate |
| NUTRISTEM ® | Lam&E- | TrypLE ™ | 5.38 × 10⁶ | 105.2 | 5.38 × 10⁷ | 5 |
| | cad | | | | | |
| ESSENTIAL 8 ® | | VERSENE ® | Split 1:14 | n/a | n/a | n/a |
| ESSENTIAL 8 ® | rVTN | Formulation 3 | 5.22 × 10⁶ | 91.0 | 2.76 × 10⁷ | 9 |
| L7 ™ | L7 ™ | Formulation 3 | 5.41 × 10⁶ | 88.9 | 2.41 × 10⁷ | 14 |
| | Matrix | | | | | |
| mTeSR ™-1 | L7 ™ | Formulation 3 | 5.21 × 10⁶ | 103.7 | 5.21 × 10⁷ | 12 |
| | Matrix | | | | | |

Based on the evaluation of multiple culture conditions and cells, similar data generated from H1 cell line (data not shown), Formulation 3 passaging formulation in combination with ESSENTIAL 8® or NUTRISTEM® was chosen for further analysis in suspension culture studies.

### Example 3

### Comparison of Formulation 3 Against Other Dissociation Treatments in Suspension Culture (3D, Biott Spinner)

H1 cells were grown in cell culture medium supplemented with different levels of bFGF in 2D culture and then transitioned into suspension culture (Biott spinner) using L7 Formulation 3 and growing in 3D in the same cell culture medium. During the cell expansion in 2D culture, the E8 medium was supplemented with basic Fibroblast grown factor (bFGF) at 100, 40 or 10 ng/mL. The cells were then removed from the culture medium and placed in 50 mL conical tubed. The vessel Biott spinner vessel was rinsed with 10 mL of DPBS and transferred to the same conical tube to transfer any residual cells. The tubes were then centrifuged at 100 g for 1 minute at room temperature to settle the cells. The supernatant was aspirated, and the cells were resuspended in 30 mL of DPBS. The cells were centrifuged again at 100 g for 1 minute at room temperature, and the supernatant was removed again by aspiration.

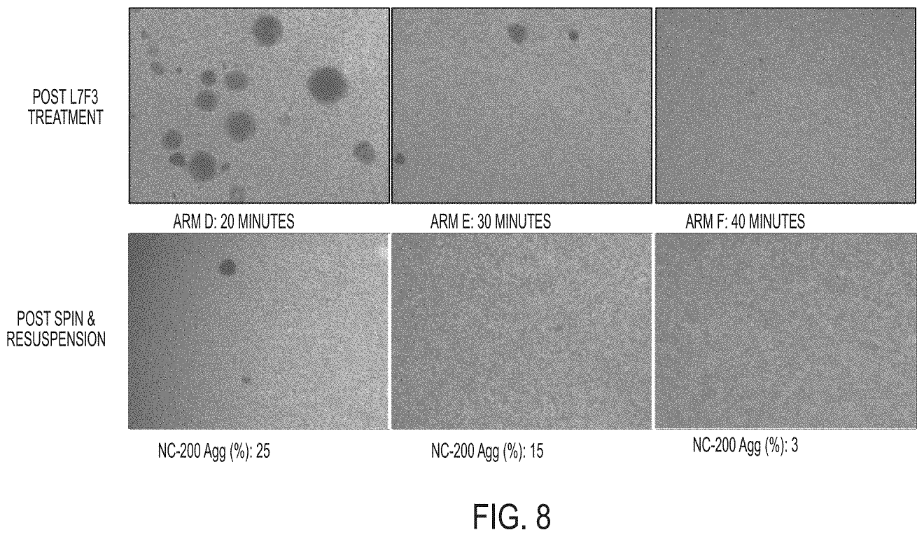

Six milliliters of pre-warmed Formulation 3 were added, and the cells were incubated in a 37° C. water bath for 15-20 minutes. The tubes were swirled every three minutes. The tube was transferred inside a BSC and the cells were pipetted 5 times with a 10 ml pipette for the entire volume. 20 mL of growth media (with Y-compound) was added to quench. The cells were then centrifuged at 200 g for 5 minutes at room temperature, aspirated to remove the supernatant, and then the cells were resuspended in 20 mL of growth media supplemented with Y compound. The total final volume was measured with a 25 mL pipetted. Cells were then counted using NC-200, one cassette method. A 10 fold dilution (450 μL of growth medium supplemented with Y compound and 50 μL of cell suspension) was performed. Cell growth and viability for the Biott spinner cultures was determined on day 4 post inoculation at 0.6×10⁶ cells/ml and presented in Table 5. The results show acceptable level of cell fold expansion (around 4-5 fold), percentage of aggregates remaining in the culture (6-12%), aggregate size (about 150-200 microns in diameter) following passaging of the cells using L7 Formulation 3. Depending on the treatment, the viability was between 80-84% for the cell treated with L7 Formulation 3. To improve the viability and reduce the percentage of aggregates remaining in the culture, further optimization of the treatment with L7 Formulation 3 was carried 3 by increasing the incubation time (from 15 min to 20, 30, and 40 min) and using agitation inside the spinner flask and results are summarized in Example 5 and FIG. 8.

TABLE 5

| | | Cell count | | Cell | | Cluster | St. Dev/ |
|---|---|---|---|---|---|---|---|
| H1 - Culture system - | Passage | (total viable) | Fold | viability | % | diameter | Min/ |
| Nutrosystem spinners | # | millions | Expansion | (% viable) | aggr. | (average) | Max |
| E8/rVTN/TrypLE ™ | 45 | 80.4 | 4.5 | 86.5 | 5 | 171.29 | 31.93 |
| 100 ng/ml | | | | | | | 96.61 |
| | | | | | | | 261.88 |
| E8/rVTN/TrypLE ™ | 45 | 85.2 | 4.7 | 97.0 | 2 | 146.67 | 23.15 |
| 40 ng/ml | | | | | | | 96.07 |
| | | | | | | | 196.03 |
| E8/rVTN/ | 45 | 74.6 | 4.1 | 83.0 | 12 | 172.15 | 43.28 |
| Formulation 3 FGF | | | | | | | 99.08 |
| 100 ng/ml | | | | | | | 338.69 |
| E8/rVTN/ | 45 | 85.5 | 4.8 | 80.5 | 12 | 171.94 | 40.81 |
| Formulation 3 | | | | | | | 110.77 |
| FGF 40 ng/ml | | | | | | | 311.46 |
| E8/rVTN/ | 45 | 74.5 | 4.1 | 84.2 | 6 | 136.6 | 42.77 |
| Formulation 3 | | | | | | | 74.82 |
| FGF 100 ng/ml | | | | | | | 378.16 |

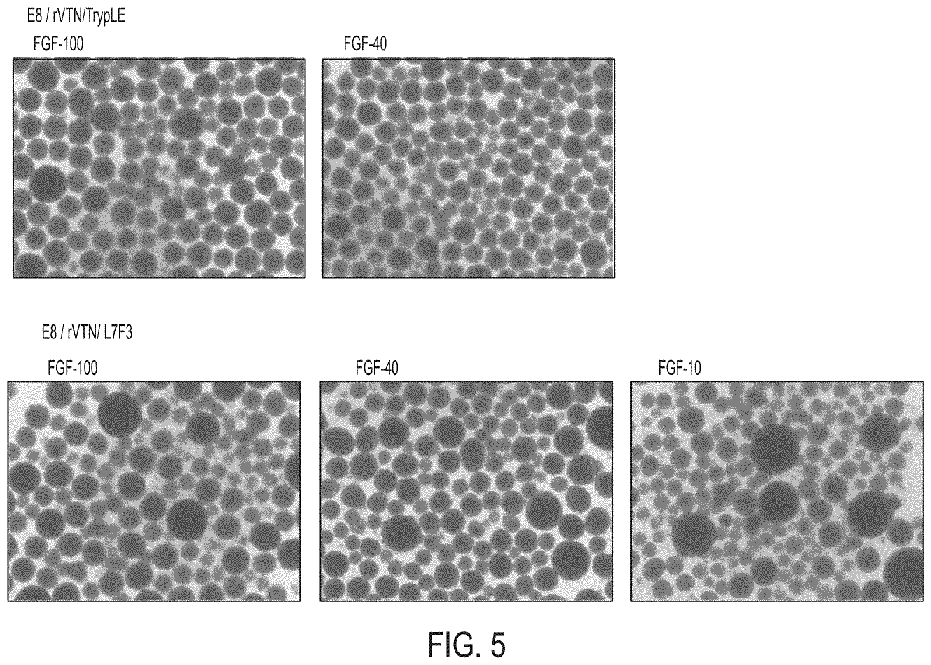

FIG. 5 shows the results of H1 cells grown in suspension culture after inoculation at a concentration of about 0.6×10⁶ cells/mL in Nutristem medium in Biott Spinner culture. Prior to inoculation in 3D Biott spinners, the cells were serially sub-cultured in 2D tissue culture flasks in (i) ESSENTIAL 8®+rVTN matrix and passaged with TrypLE™, or (ii) ESSENTIAL 8®+rVTN matrix and passaged with Formulation 3 (“L7F3”). During the cell expansion in 2D culture, the E8 medium was supplemented with basic Fibroblast grown factor (bFGF) at 100, 40 or 10 ng/mL. The cells in suspension culture were serially sub-cultured with Formulation 3 (“L7F3”) and the figure shows H1 cell aggregates on Day 4. These images demonstrate that following passaging of H1 cells using formulation 3, round and spherical aggregates of cells can be generated in suspension. The aggregate size distribution varies depending on the treatment and bFGF concentration in 2D culture. These results confirm the feasibility of serial passaging of hPSCs using L7F3 in 3D suspension culture without impacting on the growth or morphology of the cells.

### Example 4

### H1 E8 Planar Top NutriStem Biott DD1

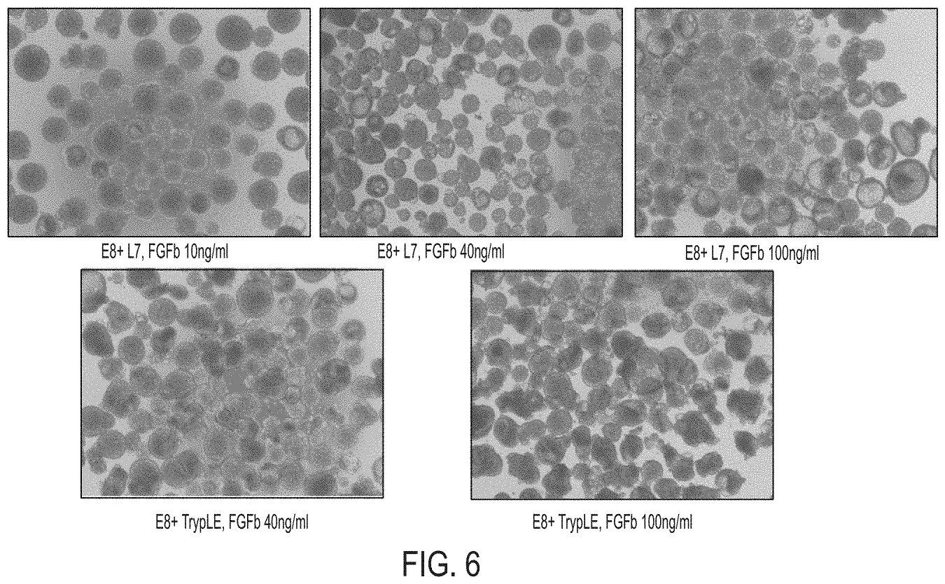

FIG. 6 shows the results of H1 cells directed differentiation into endodermal lineage based on the process depicted in FIG. 1. Following expansion in 2D (tissue culture flask) and 3D suspension culture (Biott Spinner) in different cell culture media, H1 cells serially subcultured with L7 Formulation 3 were used in this directed differentiation process (i.e. differentiation into endodermal lineage) as demonstrated by morphology of the cells resembling pancreatic progenitor cells at stage 4 of differentiation.

Table 6 shows the results of cell count viability, aggregate size and flow cytometry analysis of expression of various transcriptions factors (Oct-4, Sox-17, PDX-1, and NKX6.1) for directed differentiation of H1 cells after 3D expansion and serial subculturing using L7 Formulation 3. The cells exhibiting high level of PDX-1 and NKX6.1 (two markers used to demonstrate positive expression of pancreatic progenitor cells) and very low level of pluripotent stem cell marker Oct4 and early endoderm marker SOX-17.

| | E8/rVTN/ | E8/rVTN/ | E8/rVTN/ | E8/rVTN/ | E8/rVTN/ |
|---|---|---|---|---|---|
| | Formula 3 | Formula 3 | Formula 3 | TrypLE ™ | TrypLE ™ |
| | FGFb 100 ng/ml | FGFb 40 ng/ml | FGFb 10 ng/ml | FGFb 100 ng/ml | FGFb 40 ng/ml |
| | 37.9 | 6.52 | 36.1 | 39.1 | 20.2 |
| Cell Viability | 92.664 | 89.1 | 94.932 | 97.956 | 95.904 |
| Cluster | 186.71 ± 41.44 | 157.99 ± 48.46 | 186.54 ± 42.42 | 209.28 ± 76.17 | 200.58 ± 55.24 |
| Diameter | | | | | |
| Pdx1% | NA | 93.8 | 96.6 | 91.7 | 92.8 |
| NKX6.1% | NA | 35.8 | 62.8 | 20.8 | 30.1 |
| Sox17% | NA | 2.2 | 1.4 | 1.7 | 2.5 |
| Oct4% | NA | 9.1 | 9.1 | 12.4 | 13.5 |

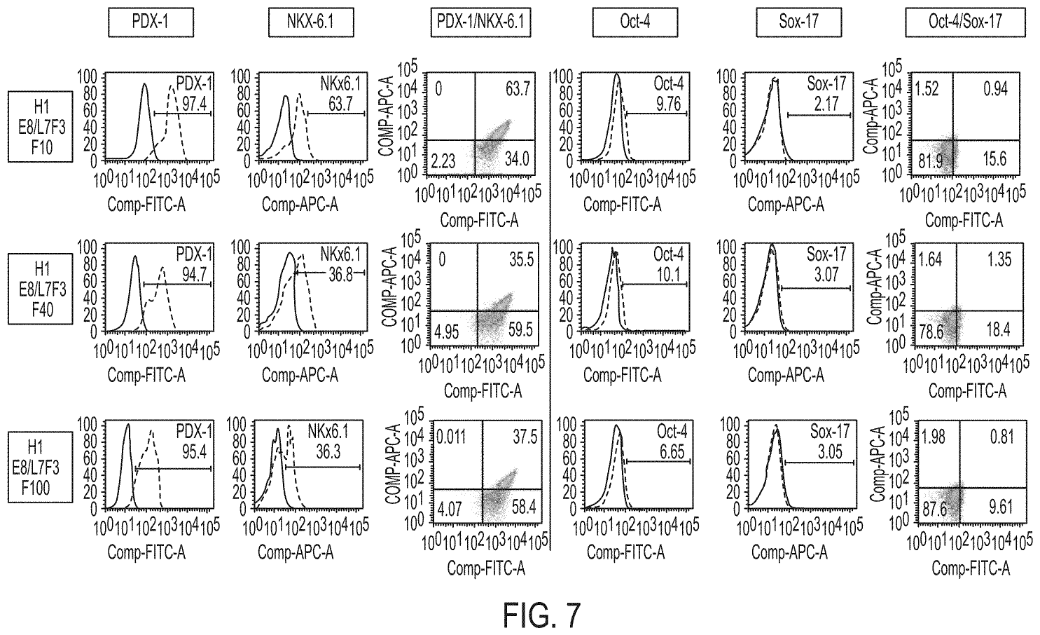

FIG. 7, Table 7, and Table 8 depicts flow cytometry analysis of expression of various transcriptions factors (Oct-4, Sox-17, PDX-1, and NKX6.1) for H1 cells following expansion in 2D (tissue culture flask), 3D suspension culture (e.g. Biott Spinner) in different cell culture media as described in FIG. 5, and then directed differentiation into pancreatic progenitor cells. The cells grown in suspension and passaged using Formulation 3 “L7F3” maintain the capacity to differentiate into high level pancreatic progenitor cells exhibiting high level of double positive expression of PDX-1 and NKX6.1 in the absence of pluripotent stem cell marker Oct4 and early endoderm marker SOX-17. Once again, the expression of PDX-1 and NKX6.1 confirms that the cells grown in suspension and passaged using Formulation 3 “L7F3” maintain the capacity to differentiate into a specific cell lineage.

TABLE 7

| | PDX-1 | NKX-6.1 | Oct-4 | Sox-17 |
|---|---|---|---|---|
| % expression | Iso/Target/Final | Iso/Target/Final | Iso/Target/Final | Iso/Target/Final |
| Formulation 3 | 0.8/97.4/96.6% | 0.9/63.7/62.8% | 0.7/9.8/9.1% | 0.8/2.2/1.4% |
| FGF-10 | | | | |
| Formulation 3 | 0.9/94.7/93.8% | 1.0/36.8/35.8% | 1.0/10.1/9.1% | 0.9/3.1/2.2% |
| FGF-40 | | | | |
| Formulation 3 | 0.9/95.4/94.5% | 1.0/36.3/35.3% | 1.0/6.7/5.7% | 0.9/3.1/2.2% |
| FGF-100 | | | | |

TABLE 8
FACS Summary

| % | PDX-1 | NKX-6.1 | Oct-4 | Sox-17 |
|---|---|---|---|---|
| Expression | Iso/Target/Final % | Iso/Target/Final % | Iso/Target/Final % | Iso/Target/Final % |
| Lam | 0.9/89.8/88.9% | 0.8/22.5/21.7% | 1.0/10.6/9.6% | 0.9/4.0/3.1% |
| FGF-10 | | | | |
| Lam | 1.0/89.5/88.5% | 1.0/18.0/17.0% | 0.9/7.0/6.1% | 1.0/3.5/2.5% |
| FGF-40 | | | | |
| Lam | 0.9/89.9/89.0% | 1.0/35.5/34.5% | 0.9/5.0/4.1% | 0.9/2.5/1.6% |
| FGF-100 | | | | |
| VTN | 1.0/91.9/90.9% | 1.0/45.0/44.0% | 0.9/8.9/8.0% | 0.8/2.9/2.1% |
| FGF-10 | | | | |
| VTN | 1.0/90.3/89.3% | 1.1/35.8/34.7% | 1.0/11.6/10.6% | 0.9/6.7/5.8% |
| FGF-100 | | | | |
| Formulation 3 | 0.8/97.4/96.6% | 0.9/63.7/62.8% | 0.7/9.8/9.1% | 0.8/2.2/1.4% |
| FGF-10 | | | | |
| Formulation 3 | 0.9/94.7/93.8% | 1.0/36.8/35.8% | 1.0/10.1/9.1% | 0.9/3.1/2.2% |
| FGF-40 | | | | |
| Formulation 3 | 0.9/95.4/94.5% | 1.0/36.3/35.3% | 1.0/6.7/5.7% | 0.9/3.1/2.2% |
| FGF-100 | | | | |
| TrypLE ™ | 1.0/93.8/92.8% | 1.1/31.2/30.1% | 0.8/14.3/13.5% | 0.9/3.4/2.5% |
| FGF-40 | | | | |
| TrypLE ™ | 0.9/92.6/91.7% | 0.9/21.7/20.8% | 0.9/13.3/12.4% | 0.9/2.6/1.7% |
| FGF-100 | | | | |

It is contemplated that a normal karyotype will be present at greater than 50 passages.

### Example 5

Passaging Pluripotent Stem Cells into Single Cell Suspension Inside a 3D Culture without Manual Pipetting

As described earlier (see example 3), further optimization of the treatment with L7 Formulation 3 was needed to improve the viability and reduce the percentage of cell aggregates remaining after dissociation with L7 Formulation 3. This goal was achieved by increasing the incubation time (from 15 min to 20, 30, and 40 min) and using agitation inside the spinner flask instead of dissociation inside a separate tube. In this case, the cells grown in the form of cell aggregates in 3D culture (500 mL spinner flasks) were settled by stopping the agitation. The medium was then aspirated using an aspirator and the cells were incubated in the spinner flask with 100 mL of Formulation 3 in 37° C. incubator for 20 minutes, 30 minutes, or 40 minutes. The cells were stirred at 70 rpm while incubating with Formulation 3. A sample was taken from each culture and the cell suspension was observed using an inverted microscope. Images were taken at different levels of the treatment (post-Formulation 3 treatment versus post-Formulation 3, spin, and resuspension) and different incubation time. FIG. 8 demonstrates the single cell suspension generated after dissociation using L7 Formulation 3 at different incubation time and different treatment level. These results show that L7 Formulation 3 can be further optimized to reduce the number of aggregates seen in the culture using 40 min incubation time. Table 9 provides a qualitative ranking of the level of aggregates observed at different incubation time, indicating 40 min incubation improved the percentage of singe cells generated with L7 Formulation 3.

TABLE 9
Aggregate Observation after Formulation 3 Treatment

| | | | Post | |
|---|---|---|---|---|
| | Formulation 3 | Post | Formulation 3 + | |
| | Incubation Time | Formulation | Post Spin and | Aggregate |
| Sample | (Minutes) | 3 Treatment | Resuspension | Ranking |
| 1 | 20 | Yes | No | Most |
| 2 | 30 | No | No | Medium |
| 3 | 40 | No | Np | Least |

The data suggests that dissociation of pluripotent stem cell aggregates in 3D suspension culture using Formulation 3 passaging solution is feasible and can be applied to large scale bioreactor systems.

What is claimed is:

1. A formulation for harvesting and passaging single cell human stem cells comprising:
(i) 1 mM to about 30 mM sodium citrate;
(ii) a salt comprising 10 mM to 170 mM KCl or NaCl; and
(iii) Ca2+/Mg2+-free Dulbecco's phosphate buffered saline (DPBS);
wherein said formulation has an osmolarity of about 100 mOsmol/liter to about 350 mOsmol/liter.

2. The formulation of claim 1, wherein the osmolarity of the formulation is of about 200 mOsmol/liter to about 300 mOsmol/liter.

3. The formulation of claim 1, wherein the osmolarity of the formulation is of about 250 mOsmol/liter to 300 mOsmol/liter.

4. The formulation of claim 1, wherein the sodium citrate is at a concentration of about 5 mMol/liter to about 15 mMol/liter.

5. The formulation of claim 1, wherein the salt is KCl.

6. The formulation of claim 5, wherein the KCl is at a concentration of about 40 mMol/liter to about 150 mMol/liter.

7. The formulation of claim 5, wherein the KCl is at a concentration of about 80 mMol/liter to about 120 mMol/liter.

8. The formulation of claim 1, wherein the formulation has a pH of about 7 to about 8.

9. The formulation of claim 1, wherein the formulation has a pH of about 7.4 and 7.8.

10. The formulation of claim 1, substantially free of enzymes.

11. The formulation of claim 1, further comprising a human stem cell.

12. The formulation of claim 11, wherein the human stem cell is selected from the group consisting of embryonic stem cell, somatic stem cell, and induced pluripotent stem cell.

13. The formulation of claim 11, wherein the human stem cell is an induced pluripotent stem cell.

14. The formulation of claim 11, wherein the human stem cell is a tissue-specific stem cell selected from the group consisting of an epidermal stem cell, blood stem cell, hematopoietic stem cell, epithelial stem cell, cardio stem cells, and neural stem cells.

15. A method for harvesting and subsequent passaging of human stem cells (hSCs) comprising:
incubating the hSCs in the formulation of any one of claims 1 to 12 in a cell culture plate or vessel for about 2 minutes to about 20 minutes, wherein said hSCs detach from the cell culture plate or vessel as single cells having cell viability of about 85% and about 100%.

16. The method of claim 15, wherein the cell culture plate or vessel is selected from the group consisting of a petri dish, multi-well cell culture plate, stacked cell culture apparatus, cell culture factory, or conical tube.

17. The method of claim 16, wherein the hSCs are incubated in a Bioreactor, 3D suspension culture vessel, or conical tube.

18. The method of claim 15, further comprising:
downstream processing of the single cells, wherein downstream processing is selected from the group consisting of continuous counter-flow centrifugation technology, formulation, automated vialing, cryopreservation, and high-throughput screening, genetic editing, and directed differentiation.

19. The method of any one of claims 15 to 18, wherein the human stem cell is selected from the group consisting of embryonic stem cell, somatic stem cell, and induced pluripotent stem cell.

20. The method of any one of claims 15 to 18, wherein the human stem cell is an induced pluripotent stem cell.

21. The method of any one of claims 15 to 18, wherein the human stem cell is a tissue-specific stem cell selected from the group consisting of an epidermal stem cell, blood stem cell, hematopoietic stem cell, epithelial stem cell, cardio stem cells, and neural stem cells.

22. A method of optimizing a formulation for harvesting and passaging single cell human stem cells, comprising:
creating a plurality of formulations for harvesting and passaging cells, each formulation comprising at least one Ca²⁺ chelator and a known osmolarity, and wherein each of the formulations in the plurality of the single-cell passaging solutions have varying concentrations and varying osmolarities,
testing each of said plurality of formulations to determine percentage of culture detached at a given treatment time and percentage of single cells at each given concentration of Ca′ chelator and osmolarity, and
selecting a preferred formulation from the plurality of formulations.

23. A single-cell passaging formulation obtained by the method of claim 22.

24. A method for harvesting and subsequent passaging of single-cell human pluripotent stem cells (hPSCs) in a 2D tissue culture vessel, comprising: passaging the hPSCs with the formulation of any one of claims 1 to 14, at a split ratio of 1:5 to 1:60, wherein the culture reaches confluence within 10 days after split.

25. A method for harvesting and subsequent passaging of human pluripotent stem cells (hPSCs) in 2D tissue culture vessel comprising:
i) plating the hPSCs in medium,
ii) aspirating the medium,
iii) washing the hPSCs with DPBS,
iv) adding the formulation of any one of claims 1 to 12 to the hPSCs and incubating for 1 minute to 30 minutes, and
v) resuspending the hPSCs in culture media.

26. The method of claim 25, wherein the formulation of (iv) is removed from the hSPCs prior to resuspending the hPSCs in culture media.

27. A method for harvesting and subsequent passaging of human pluripotent stem cells (hPSCs) grown in the form of cell aggregates in 3D suspension bioreactor comprising:
i) culturing hPSCs in the form of cell aggregates in medium using a suspension culture bioreactor,
ii) separating and removing the hPSCs from the medium,
iii) washing the hPSCs with DPBS,
iv) adding the formulation of any one of claims 1 to 12 to the hPSCs, agitating gently, and incubating for 1 minute to 50 minutes, and
v) resuspending the hPSCs in culture media.

28. The method of claim 27, wherein the formulation of (iv) is removed from the hPSCs prior to resuspending the hPSCs in culture media.
